(19) United States
(12) Patent Application Publication
(10) Pub. No.: US 2010/0021552 A1
(43) Pub. Date: Jan. 28, 2010

(54) POLYSACCHARIDE NANOFIBERS HAVING ANTIMICROBIAL PROPERTIES

(75) Inventors: Thomas Rupert Hayes, Devon (GB); Bo Su, Bristol (GB)

Correspondence Address: CONVATEC INC., 100 HEADQUARTERS PARK DRIVE, SKILLMAN, NJ, 08558, US

(73) Assignee: CONVATEC TECHNOLOGIES INC., Reno, NV (US)

(21) Appl. No.: 12/471,738

(22) Filed: May 26, 2009

(30) Foreign Application Priority Data
May 23, 2008 (GB) 0809499.7

(51) Int. Cl. A61K 9/14; B29C 47/08; B29C 35/06; A61K 33/38; A61P 31/00

(52) U.S. Cl. 424/499; 264/465; 264/477; 424/618

(57) ABSTRACT

Polysaccharide nanofibers having anti-microbial properties, said nanofibers comprising an alginate and having silver nanoparticles dispersed throughout the nanofibers.

The present invention relates to polysaccharide nanofibers having antimicrobial properties and a method of making them. In particular, the invention relates to polysaccharide nanofibers having silver nanoparticles dispersed throughout the fibers. The fibers may be produced by electrospinning and may be used in wound care.

### BACKGROUND OF THE INVENTION

The incorporation of silver into fibrous wound dressings is known. Generally, the silver is held on the surface of the fibers or dressing. Although this imparts antimicrobial properties to the dressing, it can lead to several disadvantages. Excess silver may need to be used because, due to its presence at the surface, the silver may be released or made inactive quickly. The excess, while providing a reservoir, can result in an unacceptable physical appearance of the dressing due to discoloration of the silver, or may result in staining of the skin of the patient. The incorporation of particles into fibrous wound dressings has been described in U.S. Pat. No. 7,229,689 but the method of incorporation involves the addition of silver from an ion exchange resin in order to avoid discoloration. It would be desirable to use silver in a fibrous dressing in such a manner that the silver is distributed evenly through the fibers so that a sustained release of silver is obtained from the dressing. It would also be desirable to use silver in the form of nanoparticles as silver nanoparticles have been shown to possess antimicrobial properties and present a larger surface area for release.

WO 2005/073289 discloses the mixing of metal particles with a polymer dope, prior to extrusion and solidification into fibers or films. One of the problems associated with the incorporation of nanoparticles into fibers is the difficulty of dispersing the particles uniformly as particles tend to agglomerate.

Electrospinning is a well known fabrication technique, which can be used to produce polymer fibers in the range 1 nm to 1 μm. The process of electrospinning polymer solutions involves the formation of an electrically charged liquid jet from the surface of a polymer solution in the presence of an electric field. The liquid jet undergoes stretching effects and drying as the solvent evaporates, and is deposited as polymer fiber on a suitably positioned, oppositely charged target. These electrospun polymer nanofibers are most commonly deposited in the form of a non-woven web.

In the past, relatively few natural polymers were successfully electrospun into nanofibers. Whereas synthetic polymers can have carefully controlled molecular weight and molecular weight distribution and are typically produced with long, flexible, linear chains, natural polymers are generally more complex and have strong hydrogen bonding, which leads to relatively low chain flexibility. This often results in natural polymers with unfavorable conformations.

### SUMMARY OF THE INVENTION

We have found that it is possible to produce polysaccharide nanofibers with antimicrobial properties. In particular, we have found that it is possible to incorporate silver particles into polysaccharide nanofibers.

Accordingly, a first aspect of the present invention provides polysaccharide nanofibers having antimicrobial properties, said nanofibers comprising, for example, alginate and having silver nanoparticles dispersed throughout the fibers.

Such fibers have the advantage that they present a large surface area for delivery of silver to a wound. They may also have the advantage that the silver is released to the wound in a sustained manner. By the term dispersed throughout the fiber is meant that the nanoparticles are distributed within the fibers. The particles may be distributed through the whole thickness of the fiber and, preferably, are uniformly distributed. In this way a predictable dosage of silver may be delivered to the wound.

By the term nanoparticle is meant a particle having a diameter of from 1 nm to 100 nm, generally between 1 nm-50 nm and preferably between 1 nm-10 nm.

By the term nanofiber is meant a fiber having a diameter of less than 1 micron, generally between 1 nm-500 nm, preferably between 20 nm-500 nm.

Preferably, the silver particles are present in the fibers at a concentration of between 0.002% (w/w) and 2% (w/w), more preferably between 0.02% (w/w) and 1% (w/w).

The polysaccharide nanofibers are preferably gel forming fibers, by which is meant that the fibers are hygroscopic fibers which upon the uptake of wound exudate become moist, slippery or gelatinous and thus reduce the tendency for the surrounding fibers to adhere to the wound. The gel forming fibers can be of the type which retain their structural integrity on absorption of exudate or can be of the type which lose their fibrous form and become a structureless gel. The gel forming fibers may comprise, in addition to alginate, sodium carboxymethylcellulose, pectin, chitosan, hyaluronic acid, or other polysaccharides. The gel forming fibers preferably have an absorbency of at least 2 grams of 0.9% saline solution per gram of fiber (as measured by the free swell method). Preferably, the gel forming fibers have an absorbency of at least 10 g/g as measured in the free swell absorbency method, more preferably between 15 g/g and 25 g/g.

Alginate is a natural polysaccharide existing widely in many species of brown seaweeds. The alginate for use in the present invention can be sodium alginate of the type containing a high proportion of guluronate but can also be of the type containing a high proportion of mannuronate.

The polysaccharide nanofibers may be produced by electrospinning. We have found that polysaccharide nanofibers produced by electrospinning advantageously may have silver nanoparticles uniformly dispersed throughout the fibers. The distribution can be measured by transmission electron microscopy.

A second aspect of the invention relates to an aqueous solution for spinning polysaccharide nanofibers, said solution comprising, for example:
from 2% (w/w) to 8% (w/w) of sodium alginate;
from 0.05% (w/w) to 5% (w/w) of water soluble polymer; and
from 0.00015% (w/w) to 0.2% (w/w) of silver compound.

Preferably, the solution contains from 0.1% by weight to 1% by weight of a water soluble polymer such as polyethylene oxide, polyvinyl alcohol or polyvinyl pyrrolidone or a mixture thereof. More preferably, the water soluble polymer has a long-chain linear structure and high molecular weight.

The solution may also comprise from 2% by weight to 20% by weight of a polar aprotic solvent such as DMSO to break down hydrogen bonding within the polysaccharide and improve the polymer chain entanglement during electrospinning. The solution may also comprise from 0.01% w/w to 1% w/w of non-ionic surfactant such as Triton X-100 to alter the surface tension of the solution.

Preferably, the aqueous solution of sodium alginate has a weight proportion of PEO to alginate ratio between 2% and 25% and a DMSO concentration between 5% (w/w) and 10% (w/w), with small concentrations of silver nitrate. Advantageously, silver nanoparticles can be formed in-situ in such a solution by photochemical reduction of a silver compound such as silver nitrate. Silver nanoparticles are formed when silver ions dissociate from a silver compound when it is dissolved, and gain an electron in an oxidation-reduction reaction with a reducing agent such as carboxyl and/or hydroxyl groups of polymers. This results in silver atoms which act as seeds onto which other silver ions are reduced, resulting in clusters of silver atoms which grow into nanoparticles as more silver accumulates and clusters join together. These solutions can then be electrospun to form nanofibers with diameters in the range 1 nm-1 μm, which desirably contain a uniform distribution of silver nanoparticles.

Accordingly, a third aspect of the invention relates to a process for forming polysaccharide nanofibers by:
a) making a solution comprising, for example:
   from 2% (w/w) to 8% (w/w) of sodium alginate;
   from 0.05% (w/w) to 5% (w/w) of water soluble polymer; and
   from 0.00015% (w/w) to 0.2% (w/w) of silver compound; and
b) electrospinning the solution to form nanofibers.

The electrospun nanofibers may then be ionically cross-linked in a bath containing excess calcium ions, in order to transform some or all of the sodium alginate to calcium alginate. The calcium alginate or sodium/calcium alginate nanofibers, containing silver nanoparticles may then be soaked in water to remove the excess calcium, before being dried. Preferably, the dried fibers comprise calcium alginate and sodium alginate in the ratio of 80% calcium alginate to 20% sodium alginate.

Preferably, the solution is prepared in ambient light and then stored in the dark prior to electrospinning within 12 hours of preparation, more preferably within 6 hours of preparation and more preferably within 4 hours of preparation.

Preferably, the solution has a viscosity prior to spinning of between 1 Pa:s and 10 Pa:s.

More preferably, the solution comprises an anti-agglomeration agent such as a non-ionic triblock copolymer or an organoalkoxysilane.

### DESCRIPTION OF THE DRAWINGS

FIG. 1a is a graph of UV-visible spectra showing the development of silver particles in alginate solution containing 5 mmol.L⁻¹ AgNO₃.

FIG. 1b is a graph showing growth of the 450 nm peak for alginate solutions containing a range of AgNO₃ concentrations both in ambient light conditions.

FIG. 2 is an EDX spectrum of a silver nanoparticle within the alginate fibers.

FIG. 3a ; is a TEM image of electrospun alginate nanofibers containing silver particles electrospun after 7 days.

FIG. 3b is a TEM image of electrospun alginate nanofibers containing silver particles electrospun within 4 hours of preparation (micron bars: 200 μm).

FIG. 3c is a higher magnification TEM image of an electrospun alginate nanofibers containing silver particles electrospun of FIG. 3b (micron bar: 500 μm).

FIG. 4a is an image of electrospun alginate discs without silver, on nutrient agar plates covered by a lawn of s. aureus.

FIG. 4b is a close up image of an electrospun alginate disc without silver, on nutrient agar plates covered by a lawn of s. aureus.

FIG. 4c is an image of electrospun alginate discs containing silver nanoparticles, on nutrient agar plates covered by a lawn of s. aureus.

FIG. 4d is a close up image of an electrospun alginate disc containing silver nanoparticles, on nutrient agar plates covered by a lawn of s. aureus.

### DETAILED DESCRIPTION OF THE PREFERRED EMBODIMENTS

The invention will now be illustrated by the following non-limiting examples.

### Example 1

PEO (Mw: >5000000 g.mol⁻¹) was dissolved in deionized water to a concentration of 1% -4 % (w/w). The solution was stirred until it appeared homogenous. After allowing time for degassing, a calculated mass of the PEO solution was mixed into a known mass of a solvent consisting of DMSO and deionized water, with a DMSO concentration between 2% (w/w) and 20% (w/w), preferably between 5% (w/w) and 10% (w/w). Sodium alginate was then slowly added to a vortex in the PEO/water/DMSO solution such that the total polymer concentration in the solution was between 3% (w/w) and 8% (w/w), preferably between 5% (w/w) and 6% (w/w) and the PEO to alginate ratio was between 2% and 10% by weight, preferably between 2% and 5% by weight. The solution was stirred thoroughly until it was consistently viscous and homogenous. Additions of the surfactant Triton X-100 were made, using a micropipette to a vortex in the alginate solution, such that the concentration was varied between 0.1% (w/w) and 1% (w/w).

In another solution, the deionized water was partially or entirely substituted for a dilute solution of AgNO₃, before the alginate was added, such that the AgNO₃ concentration in the alginate solution was between 0.1 mmol.L and 10 mmol.L⁻¹.

In another solution, a known volume of a 0.1 mol.L⁻¹ aqueous solution of AgNO₃ was added to the alginate using a micropipette, such that the final concentration of AgNO₃ in the alginate solution was between 0.1 mmol.L⁻¹ and 10 mmol.L⁻¹.

In another solution, PEO (Mw 600,000-1,100,000 g.mol⁻¹) was used instead of PEO (Mw: >5 000000 g.mol⁻¹). In this solution the proportion of PEO to alginate ratio used was in the range 10% to 40% by weight, preferably 15% to 25% by weight.

These solutions were either centrifuged for 3 mins to 10 mins at 2000 rpm to 4000 rpm to remove air bubbles from the solution, or they were simply left until the solutions were clear of bubbles.

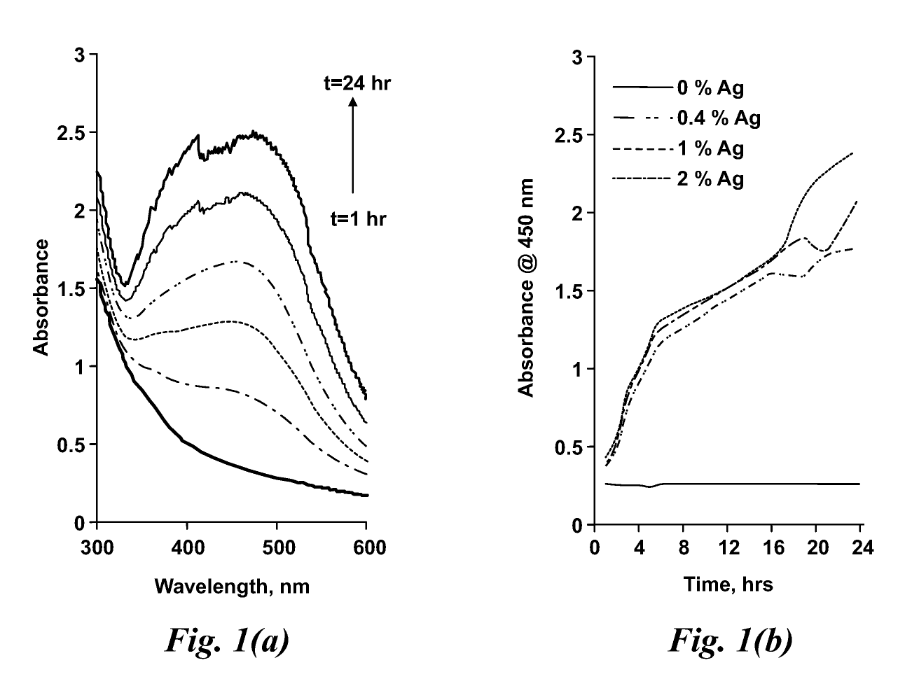

It was found that as soon as silver nitrate was mixed into the polymer solution in ambient light conditions, a reduction reaction took place. This caused a color change in the solution, from the clear yellow of an alginate solution to a dark pink or grey over time. The results of spectrophotometry confirmed these observations and can be seen in FIG. 1. It can be seen that over the first four hours after preparation of the silver containing solutions, the absorbance increases rapidly. From then on the rate of increase is reduced. The development of multiple peaks and a broadening of the peak in FIG. 1b indicate that as time progresses the silver particles grow and become aggregated.

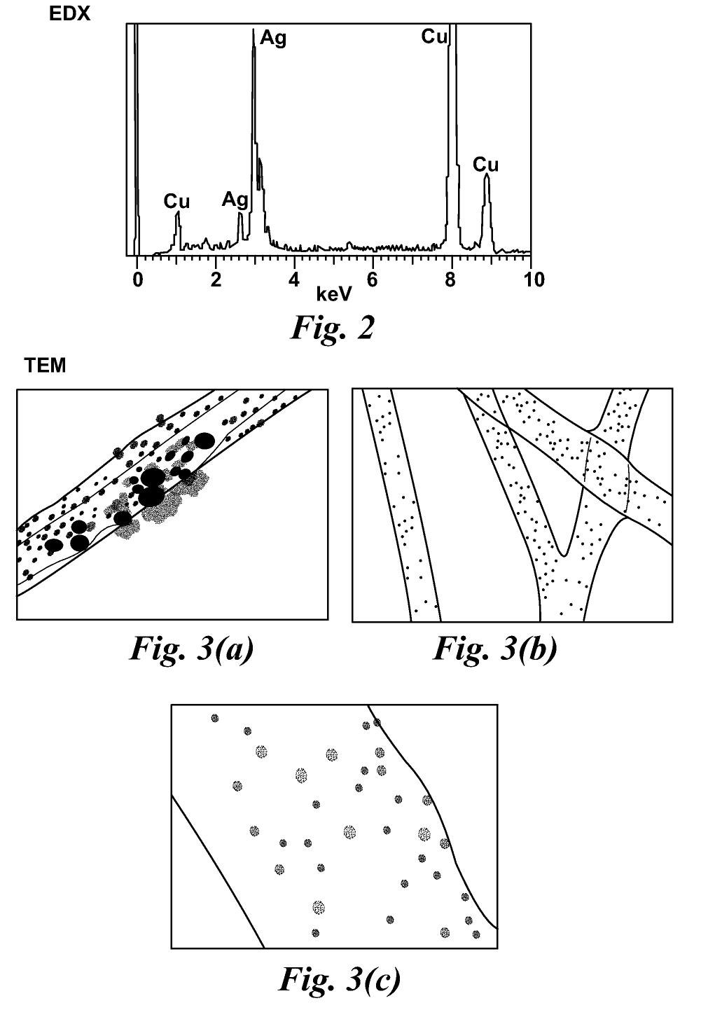

The effect of solution aging time, that is to say the time between preparation and electrospinning, on the morphology and distribution of silver particles in the alginate fibers can clearly be seen in FIG. 2. The sample produced 7 days after solution preparation has large aggregated silver particles, non-uniformly distributed, whereas the sample electrospun from fresh solution contains more evenly distributed silver particles, which are significantly smaller. With shorter solution aging times the aggregation of silver nanoparticles is reduced. It has also been found that if solutions are stored in the dark after an initial one hour aging time, particle growth and aggregation is inhibited so that alginate fibers with uniformly distributed silver nanoparticles can more easily be produced.

The alginate solutions were electrospun from a stainless steel needle of gauge size between 22 G and 31 G, which was connected to a syringe. Solution was maintained at the tip of the needle by means of a digitally controlled syringe pump, such that the flow rate was in the range 10-30 μl.min⁻¹. An applied voltage in the range 5 kV to 30 kV, preferably 10 kV to 20 kV was applied to the needle, which was positioned between 10 cm and 50 cm, preferably between 15 cm and 25 cm away from the collector.

After electrospinning, nanofibrous webs were removed from the collector and ionically cross-linked in a bath either containing an aqueous solution of CaCl₃, an organic solution of CaCl₃ followed by an aqueous solution of CaCl₃, or an aqueous organic solution of CaCl₃. After cross-linking, the fibers were soaked in either deionized water, or a mix of water and organic solvent, in order to remove any excess CaCl₃ or resulting NaCl from the fibers. Samples were then dried before characterization.

The electrospun alginate samples were characterized using scanning electron microscopy (SEM), transition electron microscopy (TEM) and energy-dispersive X-ray spectroscopy (EDX). Samples, taken for SEM before and after cross-linking, were mounted on aluminium stubs and sputter coated with 10 nm Pt/Pd before imaging. TEM samples were collected on carbon coated copper grids during electrospinning. (See FIGS. 3a, 3b and 3c.)

In order to test for antimicrobial efficacy, samples of the cross-linked alginate fibers with and without silver nanoparticles were punched into 8 mm diameter disks and sterilized in 100% ethanol before use.

Staphylococcus aureus, a common wound pathogen, was grown in nutrient broth overnight and then used to inoculate nutrient agar plates, to create a lawn of bacteria. The sample discs were then placed onto the agar plates and incubated at 37° C. for approximately 15 hrs. In this time, the lawn of s. aureus grew to form visible colonies on the agar plates. Inhibition of the growth of these colonies around the sample discs is an indicator as to the antimicrobial efficacy of the material.

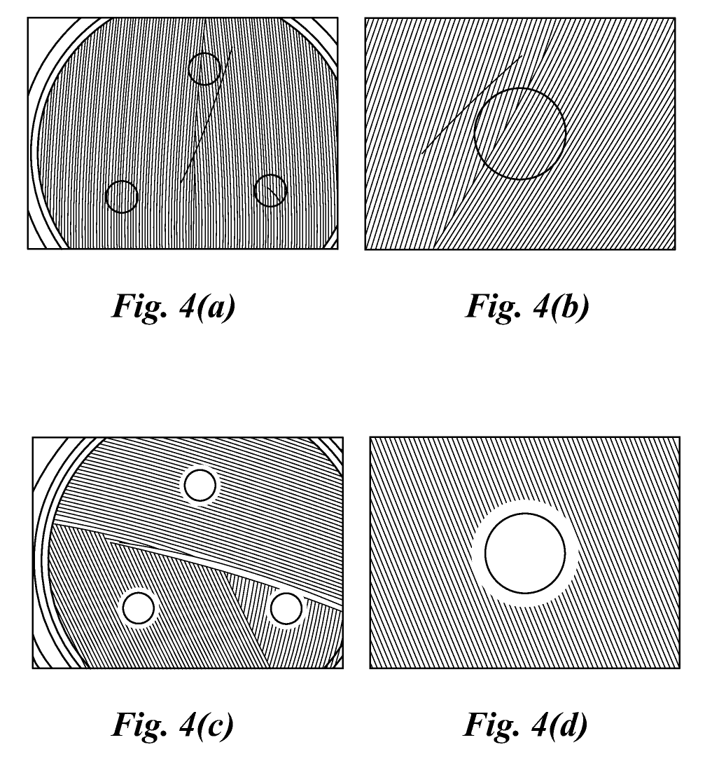

Results of the antimicrobial sensitivity assay can be seen in FIGS. 4a, 4b, 4c and 4d. It is clear that the electrospun alginate samples have no inhibitory effect on the growth of the s. aureus colonies, whereas the samples containing silver nanoparticles all inhibited the growth of the bacterial colonies directly under the discs as well as in zone around the discs.

The electrospun webs were also characterized for release into water and Solution A. Solution A is an aqueous solution with physiological concentrations of sodium chloride and calcium chloride. The release rate was found to reduce after three or four days of immersion in Solution A although even after two weeks, silver was being released. This demonstrates the desirable sustained release of silver from electrospun alginate webs.

### Example 2

The second example describes the addition of a stabilizing agent in the process described above, which restricts the growth of the silver nanoparticles and prevents them from aggregating. This allows nanofibers to be electrospun over a range of time periods, without losing the uniform distribution of fine silver nanoparticles.

The stabilizing agent used is an aqueous amphiphilic tri-block copolymer consisting poly(ethylene oxide)-poly(propylene oxide)-poly(ethylene oxide) blocks. This copolymer is capable of forming micelles around metallic nanoparticles, stabilizing them as a colloid in the aqueous solution.

We claim:

1. Polysaccharide nanofibers having anti-microbial properties, said nanofibers comprising an alginate and having silver nanoparticles dispersed throughout the nanofibers.

2. The polysaccharide nanofibers as claimed in claim 1, wherein the silver nanoparticles are uniformly dispersed throughout the nanofibers.

3. The polysaccharide nanofibers as claimed in claim 1, wherein the nanofibers are less than 1 micron in diameter.

4. The polysaccharide nanofibers as claimed in claim 1, wherein the nanofibers are between 1 nm and 999 nm in diameter.

5. The polysaccharide nanofibers as claimed in claim 1, wherein the silver nanoparticles are from 1 nm to 100 nm in diameter.

6. The polysaccharide nanofibers as claimed in claim 1, wherein the silver nanoparticles are from 1 nm to 50 nm in diameter.

7. The polysaccharide nanofibers as claimed in claim 1, wherein the silver nanoparticles are present in the nanofibers at a concentration of between 0.002% (w/w) and 2% (w/w).

8. The polysaccharide nanofibers as claimed in claim 1, wherein the nanofibers are gel forming fibers.

9. The polysaccharide nanofibers as claimed in claim 1, wherein the nanofibers are produced by electrospinning.

10. The polysaccharide nanofibers as claimed in claim 1, wherein the nanofibers are produced by electrospinning a solution comprising an alginate, a water soluble polymer and silver nanoparticles.

11. A wound dressing comprising polysaccharide nanofibers as claimed in claim 1.

12. An aqueous solution for spinning polysaccharide nanofibers, said solution comprising:
from 2% (w/w) to 8% (w/w) of sodium alginate;
from 0.05% (w/w) to 5% (w/w) of water soluble polymer; and
from 0.00015% (w/w) to 0.2% (w/w) of silver compound.

13. The aqueous solution as claimed in claim 12, wherein the solution further comprises from 2% by weight to 20% by weight of a polar aprotic solvent.

14. A process for forming polysaccharide nanofibers, said process comprising the steps of:
(a) making a solution comprising from 2% (w/w) to 8% (w/w) of sodium alginate;
from 0.05% (w/w) to 5% (w/w) of water soluble polymer; and
from 0.00015% (w/w) to 0.2% (w/w) of silver compound; and
(b) electrospinning the solution to form nanofibers.

15. The process as claimed in claim 14, wherein the solution is electrospun before the silver nanoparticles have agglomerated.

16. The process as claimed in claim 14, wherein the solution is electrospun within 12 hours of making the solution.

17. The process as claimed in claim 14, wherein the silver solution comprises an anti-agglomerating agent.

18. The process as claimed in claim 14, wherein the process comprises the further step of soaking the alginate nanofibers in a solution comprising a source of calcium ions.

19. The process as claimed in claim 18, wherein the process further comprises the step of washing the nanofibers in water.
